(19) United States
(12) Patent Application Publication
(10) Pub. No.: US 2009/0117406 A1
(43) Pub. Date: May 7, 2009

(54) CONCENTRATE FOR FILM PRESERVATION

(76) Inventor: Andrea Wingenfeld, Lauben (DE)

Correspondence Address: INTERNATIONAL SPECIALTY PRODUCTS;WILLIAM J. DAVIS, ESQ., 1361 ALPS ROAD, WAYNE, NJ, 07470, US

(21) Appl. No.: 12/300,170

(22) PCT Filed: May 11, 2007
(86) PCT No.: PCT/EP07/04219

§ 371 Date: Nov. 10, 2008

(30) Foreign Application Priority Data
May 11, 2006 (DE) 10 2006 022 257.1
May 12, 2006 (DE) 10 2006 022 631.3
Jun. 3, 2006 (DE) 10 2006 026 106.2

(51) Int. Cl. B32B 9/04; A01N 43/653; A61K 31/425; A01N 43/66; A01N 55/02; A01P 3/00; A01P 13/00

(52) U.S. Cl. 428/704; 514/383; 514/365; 514/241; 514/188

(57) ABSTRACT

The invention refers to a concentrate, in particular for the film preservation, a coating system as well as especial employment of a combination of active ingredients in the film preservation or as preservative. The concentrate contains chlorothalonile as well as one or more triazole(s) and/or isothiazolone(s).

The invention refers to a concentrate, in particular for the film preservation, a coating system as well as the especial employment of a combination of active ingredients in the firm preservation or as preservative.

Colours, plasters or also paints are commonly described as dry film systems or coatings. Coatings of this type are affected by microorganisms. These are essentially fungus and algae. For example, in the outdoor air, in particular in summer, high concentrations of mould fungus could be detected. Microorganisms could find conditions on facade coatings which make a transition to the vegetative phase possible. Plasters and colours with organic ingredients, such as for example binders, softening or thickening agents, offer in connection with humidity and with algae, together with light, favourable substrates. Different microorganisms here take root on the surfaces. These microorganisms do not, as a rule, destroy the coatings but they have the effect that the surfaces will get an unappealing black-grey-green-brown look. The affection by fungus and/or algae can be prevented by adding fungicides against fungus and algaecides against algae to the coating materials.

Film preservations for the above-mentioned coating systems are known in order to increase, for example, the permanence of colours, plasters or even paints accordingly. The employed preservatives have the aim to act as fungicide and/or algaecide, and thus to delay as long as possible that fungus or algae take roots, and to increase the look of colours, plasters paints and so on.

In the state of the art different concentrates for the film preservation of coating systems are known, such as for example, octylisothiazolone.

Accordingly equipped concentrates have a certain effect. An increasing of the efficiency by increasing the concentration is not possible as the agents are rather expensive, and the end product then may be hard to sell and cannot even be sold at all. An increasing of the concentration would also bring about undesired increasing of the toxicity for the environment and/or the user so that this does not speak either in favour of an increasing of the concentration.

Therefore it is an object of the invention to provide a concentrate as additive for the film preservation of coating systems, such as for example colours, plasters or paints, which can be produced as efficiently and also economically as possible.

On order to solve this problem the invention comes from a concentrate for the film preservation of coating systems, such as for example colours, plasters or paints, containing chlorothalonile as well as one or more triazole(s) and/or isothiazolone(s).

The complete chemical name of chlorothalonile is 1,3-dicyano-2,4,5,6-tetrachlorbenzole or tetrachloroisophthalodinitrile, the CAS number is 1897-45-6.

This agent is known as fungicide. It is employed, for example, in agriculture (vine culture, wheat, potato and so on). Chlorothalonile is also known as a wood preservative. The range of employment of a wood preservative, however, is explicitly not comprised by the striven for range of protection of this invention as the field of employment of a wood preservative refers to wood, however, the field of employment according to the invention to film preservation, in particular of corresponding coating systems such as colours, plasters or paints, which are usually applied to wood, plaster or walls. The invention refers in this respect unambiguously to a preservation or film preservation of the coating system.

According to the invention it is now suggested that chlorothalonile is combined with either one or more triazole(s) and/or isothiazolone(s). Triazole(s) or isothiazolone(s) are commonly known as fungicides. In combination with chlorothalonile with at least one triazole, or chlorothalonile with at least one isothiazolone, as well as, of course, in the combination of chlorothalonile with one triazole and at least one isothiazolone a synergetic effect has been observed, that means, an improvement of the effect of the combination compared with the single active ingredients.

In order to increase the spectrum of activity and thus also the efficiency of the concentrate according to the invention in a preferred modification it is suggested that the concentrate contains additionally at least one fungicide and/or algaecide. Depending on the chosen fungicide and/or algaecide the concentrate can be optimised for particular purposes of use, where there is, for example, a relevant affection of fungus or algae. The invention is here in no way limited to the fact that the concentrate can contain only one of each fungicide and/or only one algaecide, but actually one or more fungicides and/or algaecides.

In a preferred modification of the invention it is suggested that the concentrate contains additionally pyriothiones and/or carbamates and/or activated iodine compounds and/or pyridine derivatives and/or benzimidazole derivatives.

The above-mentioned chemical compound classes all belong generally to the fungicides.

The invention suggests furthermore that the concentrate contains additionally one or more triazines and/or urea derivatives. These active ingredient groups belonging to the algaecides are in the same way another ingredient of the concentrate (for example liquid or powder) according to the invention.

It has been observed here that in a suitably clever combination of the above-mentioned fungicides or algaecides broadband effects can be reached. The efficiency of a concentrate containing, besides chlorothalonile, at least one triazole and/or isothiazolone as well as one or more of the before-mentioned fungicides or algaecides, can be improved considerably.

Thus, for example, it has been found that a corresponding synergetic effect occurs with the concentrate according to the invention which contains propiconazol and/or octyliso-thiazolone and/or terbutryne. Propiconazol as well as octylisothiazolone are counted first of all as fungicides, terbutryne is seen as algaecide. Propiconazol here belongs to the chemical group of triazoles, octylisothiazolone belongs to the chemical group of isothiazolones. In a combination of these different fungicides or algaecides in special fields of employment a considerable raise of efficiency has been observed. It has to be taken into consideration here that the invention protects a combination of chlorothalonile only together with propiconazole as well as also a combination of chlorothalonile only together with octylisothiazolone. Already with this simple combination of active ingredients a considerable increase of the efficiency and thus synergy has been observed. This effect is even more increased in a combination of three agents, that is of chlorothalonile with propiconazole and octylisothiazolone.

The concentrate according to the invention furthermore comprises in preferred modifications additionally zinc pyrithione and/or dichloroctylisothiazolone and/or tebuconazole and/or iodine propinylbutylcarbamate and/or thiabendazola and/or carbendazim and/or diiodine methyltolylsulphone and/or 2-butyl-benzo[d]isothiazol-3-one.

Besides these agents, commonly described as fungicides, it is provided in another modification according to the invention that the concentrate contains additionally promethryne and/or N′-tert-butyl-N-cyclopropyl-6-(methylthio)-1,3,5-triazin-2,4-diamin and/or diuron and/or isoproturon which are counted commonly as algaecides.

In the following list the used agents described here by their trivial names are named chemically exactly, and also their CAS numbers as well as their primarily algaecide or fungicide properties and their affiliation to the chemical group is given.

Propiconazol

1-[[2-(2,4-dichlorphenyl)-4-propyl-1,3-dioxolane-2-yl]methyl] 1 H-1,2,4-triazol

60207-90-1

fungicide

triazole

Octylisothiazolone

2-octyl-2H-isothiazole-3-one

26530-20-1

fungicide

isothiazolone

Terbutryne

2-methylthio-4-tert.butylamino-6-ethylamino-1,3,5-triazine

886-50-0

algaecide

triazine

Zinc Pyritione

2-mercaptopyridine-N-oxide, zinc(II)salt

13463-41-7

fungicide

pyridine derivative

Dichloroctylisothiazolone

4,5-dichlor-2-octyl-2H-isothiazole-3-one

64359-81-5

fungicide

isothiazolone

Tebuconazole

1-(4-chlorphenyl)-4,4-dimethyl-3-(1,2,4-triazole-1-ylmethyl)-pentane-3-ol

107534-96-3

fungicide

triazole

Iodine Propinylbutylcarbamate

3-iodine-2-propinylbutylcarbamate

55406-53-6

fungicide

carbamate or activated iodine compound

Thiabendazole

2-(4-thiazolyl)-benzimidazole

148-79-9

fungicide

benzimidazole derivative

Carbendazim

2-benzimidazolylcarbamineacidmethylester

10605-21-7

fungicide

benzimidazole derivative or carbamate

Diiodine Methyltolylsulphone

p-[(diiodine methyl)sulphonyl]toluol

20018-09-1

fungicide

activated iodine compound

2-Butyl-benzo-[d]isothiazol-3-one

4299-07-4

fungicide

isothiazolone

Prometryne

2-methylthio-4,6-diisopropylamino-1,3,5-triazine

7287-19-6

algaecide

triazine

Cybutryine

N′-tert-butyl-N-cyclopropyl-6-(methylthio)-1,3,5-triazine-2,4-diamine

28159-98-0

algaecide

triazine

Diuron

N-(3,4-dichlorphenyl)-N′,N′-dimethylurea

330-54-1

algaecide

urea derivative

Isoproturone

N-(4-isopropylphenyl)-N′,N′-dimethylurea

34123-59-6

algaecide

urea derivative

N-Alkylisothiazolone

fungicide

isothiazolone

Preferably a N-octylisothiazolone is employed as N-alkylisothiazolone

N-Alkyl-1,2-Benzisothiazoline-3-One

fungicide

isothiazolone

As alkyl in the before-mentioned compound, for example, a C₁ to C₁₂ alkyl is provided, preferably an N-butyl-1,2-benzoisothiazoline-3-one is employed.

The above-mentioned list is only exemplary and not restricting for the invention. If the cited active ingredients comprise also other compounds which are not given here with reference to their CAS number or chemical name they nevertheless belong to the disclosure of the invention.

In a preferred modification of the invention a concentration of chlorothalonile of 0.1 to 50, in particular of 0.5 to 20, particularly preferred of 0.5 to 8 weight % is provided. By a share of chlorothalonile as large as possible the efficiency is increased accordingly, the data here always refer to the total weight of the concentrate according to the invention. With a high concentration of chlorothalonile also multiple concentrates, for example four times concentrates, can be realised. The user adds appropriately more solvent, for example water.

Very good results, with regard to an economic concentrate, have been found in particular in the weight range of 0.5 to 20 weight % chlorothalonile in the concentrate. As an optimum, if necessary in particular in connection with other additional active ingredients, a range of 0.5 to 8 weight % of chlorothalonile of the concentrate is seen. In certain cases of employment it is also convenient to offer concentrates with a rather small concentration of chlorothalonile, starting, for example, with 0.5 weight % in order to stay below the corresponding toxic limits in the concentrate. Then a higher dosage is carried out during employment.

The concentrate according to the invention is available, for example, as solution or also as dispersion or mixture of solids.

Therefore it is suggested according to the invention that the concentrate is provided as liquid concentrate, in particular as solution or dispersion. As solvents here, for example, glycols, alcohol, glycolether, glycolester can be used, for example sole or in mixtures.

Ideally in a dispersion, in particular in a water dispersion, a thickening agent is provided.

Besides the employment of the concentrate as part of a liquid (solution or dispersion), it is, however, also possible that the concentrate is used and provided in powder form or as solid or mixture of solids. Here the concentrate contains preferably fillers, in particular silicates, silica gel, organic carrier materials or even polyacrylates. This makes it possible that the concentrate can be transported, for example, as bulk material. Depending on the case of application then liquid in the desired dosage is added to the concentrate which is in powder form or mixture of solids, or the concentrate is added directly to the target substance which has to be preserved.

It is an advantage of the invention that it is provided as liquid as well as solid agent so that the employment is increased accordingly, that means the preservative can be applied very flexibly.

The activated iodine compounds have a very wide, interesting spectrum of activity against fungus. In particular iodine propinylbutylcarbamate is an interesting additive for the concentrate according to the invention because of this high efficiency and the favourable toxicological and environmental toxicological profile.

Basically it is convenient when additives are chosen for the concentrate according to the invention which have a high activity, however a low solubility in water and a good hydrolysis resistance with high pH values. These characteristics are, for example, combined in triazine or triazine derivatives, which are used as algaecides.

An effective protection against algae is necessary in the coating system in particular when the coating system is applied to a heat insulation composite system. Because of the constantly improving heat insulation of houses, there is the problem with facades that they remain wet during a longer period of time due to dew water, and thus algae can develop preferably. Besides the employment of triazine derivatives as algaecides it is very advantageous also to use urea derivatives so that by means of combinations of active agents synergetic effects between urea derivatives and triazines against algae can be exploited.

Film preservatives, such as, for example, the liquid concentrate must meet a number of demands which partly exclude each other. The combination of the different presented additives therefore makes it possible to provide an appropriate modification for each coating system. The preservatives are required to be highly effective, compatible with the coating material, the underground to which the coating material should be applied, little soluble in water (in order to prevent to be washed out of the coating system), to have a low vapour pressure, to be stable against hydrolysis, resistant against UV, to be sufficiently bio-available in the employment, to have low application costs and to be decomposable in the environment. In order to increase the stability or long efficiency of the concentrate according to the invention it is also common to provide stabilisers in the concentrate in order to obstruct or prevent thus evaporation or chemical decomposition of certain active agents.

Furthermore it is provided in the concentrate according to the invention that a concentration of propiconazol of 0 to 50 weight %, in particular of 0.5 to 20 weight %, preferably of 0.5 to 7 weight % is used. Propiconazol is characterised in particular by a high resistance against alkali hydrolysis.

The invention furthermore provides a modification where a concentration of octylisothiazolone is provided in an amount of 0 to 20 weight %, in particular of 0.2 to 15 weight %, preferably about 0.2 to 3 weight %. The limit of 3 weight % here comprises an interval of ±0.5 weight %.

Also isothiazolones, in particular octylisothiazolone, are interesting as additive for the concentrate according to the invention because of their high activity and their wide spectrum of activity.

Also the combination of the fungicide active agents with an algaecide active ingredient, such as for example terbutryne, widens the spectrum of activity of the concentrate according to the invention. For that purpose, for example, a constellation of terbutryne of 0 to 35 weight %, in particular of 1 to 20 weight %, preferably of 1 to 10 weight % is provided.

The concentrate according to the invention is, for example, liquid, that means it is provided as liquid phase. Preferably the concentrate is planned with a high part of water, typically 10 to 98 weight %, when the concentration of the other active agents are higher it is provided according to the invention to keep the water amount between 60 and 80 weight %. Basically it is possible to design the liquid concentrate based on another solvent, for example out of the group of alcohols, glycols or glycolethers and so on, exclusively or in combination with water.

In the following several recipes of the concentrate according to the invention and their effects are described. The following recipes are all water dispersed solutions. The first two recipes contain about 3 weight % octylisothiazolone

The first recipe contains 5 weight % propicanozol and 5 weight % chlorothalonile.

The second recipe contains 8 weight % chlorothalonile and 0 weight % propicanozol. In comparison of these two recipes, in the first recipe a higher efficiency has been detected than in the second recipe although the amounts of the active agents are almost equal.

In the third recipe a 5 times dilution compared with the first recipe is provided. That means that in the third recipe about 1 weight % chlorothalonile, about 1 weight % propiconazol, 0.6 weight % octylisothiazolone as well as about 1.6 to 2 weight % algaecides are provided. A recipe determined in this way can be transported and handled generally recognised as safe with respect to toxicological regulations. In the application case it is then dosed, for example, five times higher in order to reach the appropriate efficiency.

Based on the liquid concentrate of the first recipe a film preservation additive has been realised and tested in a coating system, in particular in a silicone plaster. A concentration of 0.2% of the film preservation additive according to the first recipe showed good, a concentration of 0.4% showed perfect efficiency compared with standard and blackening fungus. The efficiency, that means the necessary concentration of the liquid concentrate, was four times better compared with a reference product (Fungitrol TOP), which is designed of the active fungicides octylisothiazolone and propiconazol.

A similar raise in efficiency has also been reached with the employment of the first recipe as film preservation additive to a silicone colour.

In another group of recipes the recipes comprise additionally a weight amount of 8% terbutryne. By means of that the spectrum of activity is broadened accordingly against algae.

Basing on the invention tests have been carried out concerning the synergetic effect which will be described in the following.

### 1. Description of the Sample

The sample to be tested is plaster. The untreated sample is equipped with the cited biocides or single active agents in different concentrations. The completion is carried out following the method VdL guideline 06 “Fungicide Trial With Weathering”.

### 2. Preparation of the Samples

For preparing the samples the plaster is equipped with biocides and from that test pieces are formed. For that purpose for each test piece 7 g plaster are weighed in a small plastic mould and spread evenly. For complete drying the plaster test pieces are stored for three days at room temperature. The prepared test bodies are put in 100 ml tap water each for three days the water being exchanged daily. Then a 15 h phase of drying follows in a circulation air cabinet at 50° C. and 60 circles/min. After three days of equi libration at room temperature the test pieces are sterilised superficially and brought in the biological test. The superficial sterilisation is carried directly before putting them on the nutritive substratum by a exposure to UV radiation on both sides each for 10 minutes under a mercury medium pressure radiator.

The evaluations for fungus are carried out on malt extract agar (for example by Fluka). For mix 1 Alternaria alternata DSM 62010 and Ulocladium consortial DSM 6201 is used. For mix 2 Aspergillus niger DSM 1988, Penicillium expansum DSM 1282 and Trichoderma viride DSM 63064 is used. These are used as about 14 days old cultures on inclined agar tubes for producing suspension. For that purpose to each tube 3 ml sterile Ringer's solution are added, and then washed away with a sterile os (this corresponds with a germination index of about 10⁸ to 10⁹ KBE/ml [KBE=(Kolonie bildende Einheiten) units forming colonies]). From this a 10% suspension is produced in sterile Ringer's solution corresponding to a germination index of about 10⁷ to 10⁸ KBE/ml. The nutritive substratum with the put-on test pieces is injected with one of the listed fungus mixtures and incubated at room temperature. After 7, 14 and 21 days the test pieces are evaluated with respect to the fungus growth according to the following rating:

00=sample surface without fouling, with inhibitory aureole around test piece

0=sample surface without fouling, without inhibitory aureole around test piece

1=up to 90% inhibited fouling surface on the surface of test piece

2=70 to 90% inhibited fouling surface on the surface of the test piece

3=50 to 70% inhibited fouling surface on the surface of the test piece

4=<50% inhibited fouling surface on the surface of the test piece

### 3. Achieved Synergy Indices

From the different tests and samples synergy indices were determined for different combinations of active agents.

An evaluation of the synergy is done according to the method by F. C. Kull et al., Applied Microbiology, 9: 538 (1961):

Synergy index (SI)=Qₐ/Q_{A}+Q_{b}/Q_{B}+Q_{c}/Q_{C}

Qₐ=Concentration of component A in the biocide mixture with the desired effect (e.g. no growth of micro organisms)

Q_{A}=Concentration of component A as sole biocide with the desired effect

Q_{b}=Concentration of component B in the biocide mixture with the desired effect

Q_{B}=Concentration of component B as sole biocide with the desired effect

Q_{c}=Concentration of component C in the biocide mixture with the desired effect, and

Q_{C}=Concentration of component C as sole biocide with the desired effect.

Synergy of the two biocide active agents now exists when the synergy index (SI) shows a value below 1 (<1).

| | | |
|---|---|---|
| | ppm a.i. in | |
| | plaster dosed | |
| Active agent | additionally(*) | Synergy index |

Mix 1

| | | |
|---|---|---|
| CTL | 400 | |
| OIT | 200 | |
| Propiconazol | 2000 | |
| CTL + propiconazol + OIT | 100 + 100 + 60 | 0.6 |

Mix 2

| | | |
|---|---|---|
| CTL | 800 | |
| OIT | >500(#) | |
| Propiconazol | >2000(#) | |
| CTL + propiconazol + OIT | 200 + 200 + 120 | 0.59 |

(*)Minimal ppm concentration as fungus growth is obstructed clearly (rating - see above - after 21 days 0 or 1)
(#)The necessary concentrations are larger than to be expected with the given values (> . . . ) as the test results with the cited concentrations have not reached a rating of 0 or 1 after 21 days.

The order of the ppm concentration refers to the order of the active agents given in this line, CTL here indicates chlorothalonile, OIT octylisothiazone.

Furthermore, does the invention not only refer to the concentrate as described in the beginning, but it comprises in the same way also a coating system with a film preservation additive, wherein as film preservation additive a concentrate as described is employed. As coating systems here, of course, suitable colours, paints or plasters are seen. The affection of coating systems with fungus or algae is here obstructed considerably by the coating systems according to the invention. The coating systems remain clean and appealing for a longer time. The invention makes it possible here to employ a liquid concentrate in the application case of the coating system as well as also to employ the concerned concentrate in powder form or as mixture of solids.

In a preferred modification of the coating system here a concentration of the film preservation additive is provided in the range of 0.1 to 20 weight %, preferably 0.2 to 15 weight %, in particular of 0.2 to 3 weight % or 3 to 15 weight %.

The coating system is eventually a target system where the preservative according to the invention or the concentrate is employed. Basically it is necessary to employ in the target system a rather large or also a smaller concentration of the film preservation additive containing the concentrate according to the invention. The result is therefore, for example, in rather low dosed systems a concentration of 0.2 to 3 weight % of the film preservation additive. The eventual concentration of the active ingredients of the concentrate according to the invention can therefore be set through the respective recipe of the concentrate directly as well as through the selection of the concentration of the film preservation additive in the coating system according to the invention. This also offers the opportunity to select a higher concentration of the film preservation additive so that quite preferably also an interval of 3 to 15 weight % of the concentration of the film preservation additive is provided in the coating system in a modification according to the invention.

The invention comprises furthermore also the employment of chlorothalonile in combination with one or more triazolones and/or isothiazolones in the film preservation, in particular in the film preservation of coating systems such as colours, plasters and/or paints. The aspect of the employment according to the invention here does not only extend to the employment of chlorothalonile in the above cited combination, as described, but also to all other combinations around chlorothalonile with the other fungicides or algaecides, as claimed. This means that also all other modifications have to be seen also as disclosed in the employment according to the invention.

The invention also comprises the employment of chlorothalonile in combination with one or more triazoles and/or isothiazolones as preservative in sealants, for example in silicone compounds or also as preservatives in building protective agents, in particular for concrete preservation as well as preservative in systems for concrete redeveloping. These fields of application are, similar to coating systems, that means colours, plasters or paints, exposed to a permanent weathering and can be damaged correspondingly by algae or fungus growth. The employment in combination of chlorothalonile with one or more triazoles and/or isothialones leads also in these fields of application to a considerable improvement of the protection.

All cited interval limits are circa data and comprise according to the invention at least a range of ±0.5 weight %. The effect according to the invention can also be observed outside these given limits.

The claims filed with the application now and to be filed later on are attempted formulations without prejudice for obtaining a broader protection.

If here, on closer examination, in particular also of the relevant prior art, it turns out that one or the other feature may be convenient for the object of the invention, however, not decisively important, of course, already now a formulation is striven for which does not contain anymore such a feature, in particular not in the main claim.

Furthermore it has to be taken into consideration that the embodiments and modifications of the invention described in the different examples can be combined with each other at will. Here single or several characteristics can be exchanged with each other at will. These combinations of characteristics are also disclosed.

References in the sub-claims relate to the further design of the matter of the main claim through the characteristics of the respective sub-claim. These are, however, not to be understood as a waiver of independent protection of the matter for the characteristics of the referred sub-claims.

Characteristics only disclosed in the description so far may now, in the course of proceedings, be claimed as being of inventive relevance, for example to distinguish from the state of the art.

Characteristics only disclosed in the description or even single characteristics of claims comprising a number of characteristics may be taken over in the first claim at any time to distinguish from the state of the art, and this is even if such characteristics have been mentioned in connection with other characteristics, and achieve particularly convenient results in connection with other characteristics, respectively.

1. Concentrate comprising chlorothalonile as well as one or more triazole(s) and/or isothiazolone(s).

2. Concentrate according to claim 1, characterised in that the concentrate contains additionally at least one other fungicide and/or algicide.

3. Concentrate according to claim 1 characterised in that the concentrate contains additionally one or more pyriothiones and/or carbamates and/or activated iodine compounds and/or pyridine derivatives and/or benzimidazole derivatives.

4. Concentrate according to claim 1 characterised in that the concentrate contains additionally one or more triazines and/or urea derivatives.

5. Concentrate according to claim 1 characterised in that the concentrate contains propiconazol and/or octylisothiazolone and/or terbutryne.

6. Concentrate according to claim 1 characterised in that the concentrate contains zinc pyrithione and/or dichloroctylisothiazolone and/or tebuconazol and/or iodine propinylbutylcarbamate and/or thiabendazole and/or carbendazim and/or diiodine methyltolylsulphone and/or 2-butyl-benzo[d]isothiazol-3-on and/or N-alkylisothiazolones and/or N-alkyl-1-1,2-benzisothiazolin-3-ons.

7. Concentrate according to claim 1 characterised in that the concentrate contains prometryne and/or N′-tert-butyl-N-cyclopropyl-6-(methylthio)-1,3,5-triazin-2,4-diamin and/or diuron and/or isoproturon.

8. Concentrate according to claim 1 characterised by a concentration of chlorothalonile of 0.1 to 50 weight %.

9. Concentrate according to claim 5 characterised by a concentration of propiconazol of 0 to 50 weight %.

10. Concentrate according to claim 5 characterised by a concentration of octylisothiazolone of 0 to 20 weight %.

11. Concentrate according to claim 5 characterised by a concentration of terbutryne of 0 to 35 weight %.

12. Concentrate according to claim 1 characterised in that the concentrate is a liquid concentrate.

13. Concentrate according to claim 1 characterised in that as solvent glycol, alcohol, glycolether, glycolester are provided.

14. Concentrate according to claim 1 characterised by a concentration of water of 10 to 98 weight %.

15. Concentrate according to claim 12 characterised in that the liquid concentrate comprises a water dispersion comprising thickening agents.

16. Concentrate according to claim 1 characterised in that the concentrate is in powder form or solid form or a mixture of solids.

17. Concentrate according to claim 16 characterised in that the concentrate contains fillers selected from the group consisting of silicates, silica gel, organic carrier materials, polyacrylates, and mixtures thereof.

18. Concentrate according to claim 1 characterised in that the concentrate serves as preservative.

19. Coating system with a film preservation additive, characterised in that the film preservation additive comprises a concentrate comprising chlorothalonile as well as one or more triazole(s) and/or isothiazolone(s).

20. Coating system according to claim 19 characterised by a concentration of the film preservation additive in the range of 0.1 to 20 weight %.

21. Application of chlorothalonile in combination with one or more triazole(s) and/or isothiazolone(s) in the film preservation or as preservatives in sealants, or as preservative in building protective agents or as preservative in systems for concrete redeveloping.

22. Concentrate according to claim 8 characterized by a concentration of chlorothalonile of 0.5 to 20 weight %.

23. Concentrate according to claim 22 characterized by a concentration of chlorothalonile of 0.5 to 8 weight %.

24. Concentrate according to claim 9 characterized by a concentration of propiconazol of 0.5 to 20 weight %.

25. Concentrate according to claim 24 characterized by a concentration of propiconazol of 0.5 to 7 weight %.

26. Concentrate according to claim 10 characterized by a concentration of octylisothiazolone of 0.2 to 15 weight %.

27. Concentration according to claim 26 characterized by a concentration of octylisothiazolone of 0.2 to 3 weight %.

28. Concentrate according to claim 11 characterized by a concentration of terbutryne of 1 to 20 weight %.

29. Concentrate according to claim 28 characterized by a concentration of terbutryne of 1 to 10 weight %.

30. Concentrate according to claim 23 characterized by a concentration of propiconazol of 0.5 to 7 weight %.

31. Concentrate according to claim 30 characterized by a concentration of octylisothiazolone of 0.2 to 3 weight %.

32. Concentrate according to claim 14 characterized by a concentration of water of between 60 and 80 weight %.

33. Coating system according to claim 20 characterized by a concentration of the film preservative additive in the range of 0.2 to 15 weight %.

34. Coating system according to claim 33 characterized by a concentration of the film preservative additive in the range of 0.2 to 3 weight %.

35. Coating system according to claim 20 characterized by a concentration of the film preservative additive in the range of 3 to 15 weight %.

36. Coating system according to claim 19 wherein the concentrate comprises propiconazol and octylisothiazolone.
